(19) United States
(12) Patent Application Publication
(10) Pub. No.: US 2022/0050029 A1
(43) Pub. Date: Feb. 17, 2022

(54) ELECTROPHORETIC BIOLOGICAL SAMPLE STAINING METHOD AND STAINING APPARATUS USING

(71) Applicant: CRAYON TECHNOLOGIES INC., Seoul (KR)

(72) Inventors: Hweon PARK, Seoul (KR); Hoseung LEE, Gunpo-si (KR); Seunghyun LIM, Namyangju-si (KR); Myungsu NA, Seoul (KR)

(21) Appl. No.: 17/056,451

(22) PCT Filed: May 9, 2019
(86) PCT No.: PCT/KR2019/005595

§ 371 Date: Nov. 18, 2020

(51) Int. Cl. G01N 1/31

(52) U.S. Cl. CPC G01N 1/31; G01N 2001/302

(57) ABSTRACT

A biological sample staining method according to an embodiment of the present invention may include: positioning a biological sample to be adjacent to a staining reagent for a biological sample, and separating the biological sample and the staining reagent for the biological sample from an outer buffer by using an ion conductive film; and forming an electric field so that a current flows through the ion conductive film to the staining reagent for the biological sample and the biological sample. In this case, the biological sample is separated from a living body.

### TECHNICAL FIELD

### Background Art

### (a) Field of the Invention

The present disclosure relates to a biological sample staining method and staining apparatus, and more particularly, to a biological sample staining method and staining apparatus using an electrophoresis technique.

### (b) Description of the Related Art

When a thick biological tissue sample is observed with an optical device such as a microscope, there is a problem that light scattering severely occurs and a resolution is geometrically deteriorated, so it is difficult to image an inner tissue of the thick biological tissue sample. In order to overcome these limitations, tissue clearing techniques have been continuously researched.

In the cleared tissue, since large and small pores are formed inside a structure thereof, a fluorescent antibody may pass through it, so immunostaining is theoretically possible, but when a typical immunostaining method is used through traditionally used passive diffusion, an immunostaining time exponentially increases in proportion to a thickness of the cleared tissue, so that practical immunostaining is impossible. Recently, research related to this has been continued, but satisfactory results have not been achieved in terms of efficiency and effectiveness.

Immunostaining techniques for biological tissue samples that have been used or recently developed are as follows:

First, as described above, the most common tissue staining technique is the diffusion method. The diffusion method is a method in which an antibody diffuses into tissue by immersing the tissue in a solution containing an immune antibody, and is passive staining. The diffusion method is the simplest and easiest method, but when a thickness of a biological tissue sample is 50 to 100 μm or more, it takes a long time to stain the inside of the tissue only by diffusion, and it is impossible to commercialize it because it requires a high concentration of a large amount of antibodies. Second, as a technique designed to stain thick biological samples, there is a method of moving an immune antibody into a sample by using centrifugal force. As such, the method using the centrifugal force may move an antibody into a relatively thick biological sample, but damage is applied to the tissue by the centrifugal force, so there is a limitation in observing a shape of intact tissue.

Third, as a technique that performs immunostaining by forming an electric field, there are limitations in efficiency and practicality in that the highest resistance is applied to a center of a sphere due to a straightened electric field, and because of this, a large difference occurs in antibody staining between an edge and a center, making uniform staining impossible; in that a nano-pore membrane is used to limit movement of a sample, but as staining proceeds, an antibody concentration is diluted due to inflow of a solution due to osmotic pressure; in that because a frame is fixed, there are physical limitations in staining tissues of various shapes and sizes; in that since a pore size of a commercially available nano-pore membrane is not varied, a stain or buffer component with very small molecular weights flow out of a chamber; in that because a pore size of a nano-pore membrane is not constant, the reproducibility of staining is degraded; and in that a large amount of immune antibodies must be used to perform one staining.

While minimizing tissue damage, it is necessary to develop a technique that may have excellent efficiency and accessibility, that may have flexibility to be used for various types of samples, and that may immunostain biological tissues with the use of a minimum amount of immune antibodies within a minimum amount of time.

### PRIOR ART DOCUMENTS

### Non-Patent Document

(Non-patent document 1) “Structural and molecular interrogation of intact biological systems”, Chung et al., NATURE, Vol. 497 No. 6, 2013, 332-337,
(Non-Patent Document 2) “ACT-PRESTO: Rapid and consistent tissue clearing and labeling method for 3-dimensional (3D) imaging”, Lee et al., Scientific Report, Vol 18631, 2016.
(Non-Patent Document 3) “Stochastic electrotransport selectively enhances the transport of highly electromobile molecules”, Kim et al, PNAS, Vol 112 no. 46, 2015.
(Non-Patent Document 4) “Optimization of CLARITY for clearing whole-brain and other intact organ”, Jonathan et al, eNeuro, 2015

### DISCLOSURE

### Technical Problem

The present invention provides a staining method of a biological sample that uses an ion conductive film to prevent direct contact between a staining reagent for a biological sample and an electrode to prevent physicochemical denaturation and damage of the staining reagent and to prevent it from being mixed with an outer buffer to maintain a constant antibody concentration during the staining process so as to be able to effectively and quickly perform staining of a biological sample, and provides a staining apparatus of a biological sample to which the method is applied.

### Technical Solution

An embodiment of the present invention provides a biological sample staining method, including: positioning a biological sample to be adjacent to a staining reagent for a biological sample, and separating the biological sample and the staining reagent for the biological sample from an outer buffer by using an ion conductive film; and forming an electric field so that a current flows through the ion conductive film to the staining reagent for the biological sample and the biological sample. The biological sample is separated from a living body.

The forming of the electric field may include forming an electric field so that a current sequentially flows in an electrode of the same polarity as the staining reagent for the biological sample, the ion conductive film, the staining reagent for the biological sample, the biological sample, and an electrode of opposite polarity to the staining reagent for the biological sample in a forward direction, a reverse direction, or both directions.

The forming of the electric field may include applying a voltage to flow a current of 60 to 100 mA for 1 to 5 hours.

The applying of the voltage may be performed so that a direction of the current is changed at intervals of 5 to 60 minutes.

The biological sample staining method may further include, after the applying of the voltage, leaving it for 10 minutes to 2 hours.

After the leaving, washing may be further performed for 1 to 3 hours.

The ion conductive film may include a cation selective permeation electrolyte membrane.

The staining reagent for the biological sample may be a target binding protein or a target binding nucleic acid molecule.

The staining reagent for the biological sample may be labeled with a fluorescent label.

The biological sample may be a tissue with a thickness of 0.1 mm to 10 mm.

The biological sample may be a sample fixed by using formaldehyde (HCHO).

The biological sample may be a tissue clearing sample containing CUBIC and CLARITY.

The biological sample staining method may further include measuring a signal generated by the staining of the biological sample.

The biological sample staining method may further include cooling.

The cooling may include exchanging an electrode buffer, circulating cooling water outside the ion conductive film, or both.

The biological sample staining method may further include recovering a staining reagent for an unreacted biological sample.

Another embodiment of the present invention provides a sample chamber including: a sample chamber frame including a staining reagent part for a biological sample, a biological sample fixing part, and a buffer part that are arranged in a first direction in an inner space opened in the first direction; a biological sample holder that is able to be fixed to the biological sample fixing part and is able to contain a biological sample; and an ion conductive film that separates the inner space from the outside by being fixed to a portion of the sample chamber frame corresponding to the inner space outside the sample chamber frame.

The biological sample holder may include a holder body having a hole therein and a mesh positioned at both sides of the hole.

The sample chamber may further include a film fixing plate that presses and fixes the ion conductive film toward the sample chamber frame to a pair of sides of the sample chamber frame facing each other in the first direction.

The ion conductive film may be fixed to the sample chamber frame by interposing a gasket for film sealing in front and rear directions according to the first direction.

A volume of the staining reagent part for the biological sample may be formed to be larger than that of the buffer part.

Another embodiment of the present invention provides a biological sample staining apparatus, including: the sample chamber; and an electrode part including a first electrode and a second electrode positioned outside a pair of sides of the sample chamber facing each other in the first direction.

The biological sample staining apparatus may further include an outer chamber into which the sample chamber is inserted into an opening that is divided into a first space and a second space by a horizontal wall and is partially opened in a middle portion of the horizontal wall, wherein the first electrode may be positioned in the first space, and the second electrode may be positioned in the second space.

The biological sample staining apparatus may further include a buffer inlet that is connected to each of the first space and the second space of the outer chamber and is positioned at a lower end portion of the outer chamber, and a buffer outlet that is connected to the outside from each of the first space and the second space of the outer chamber and is opened upward from an upper end portion of each of the spaces.

### Advantageous Effects

According to the staining method of the biological sample provided in the present specification, while moving a staining reagent (for example, an antibody) for a biological sample to a thick tissue sample by using electric force, it is possible to use a sample chamber with an ion conductive film to prevent leakage of all organic molecules having extremely low electrical resistance and including macromolecules.

In addition, it is possible to provide a sample chamber of which a shape may be freely implemented according to a type and size of a biological sample, in which no osmotic pressure phenomenon occurs, and in which unnecessary heat is not generated during electrophoresis to have high electrical efficiency.

Further, since an amount of electricity applied from the electrode is almost the same as an amount of electricity applied to a sample by using an ion conductive film with almost no electrical resistance, the electric force applied to the sample may be accurately measured, thereby realizing high reproducible and efficient staining.

Further, it is possible to accelerate movement of an antibody by using electric force to speed up staining of a sample, thereby enabling efficient staining.

Furthermore, it is possible to minimize degeneration of an antibody responsible for staining and damage to a stained biological sample tissue, by cooling around an ion conductive film.

That is, according to the biological sample staining technique provided in the present specification, it is possible to stain the inside of a thick biological sample, to remarkably shorten a time required for staining a biological sample, and to effectively stain a biological tissue even when a small amount of staining reagent for a biological sample is used.

### DESCRIPTION OF THE DRAWINGS

FIG. 1 schematically illustrates a view of a process of staining a biological sample using an electrophoretic method according to an embodiment.

FIG. 2A illustrates a graph of a voltage to a time in a typical electrophoretic voltage application method, and FIG. 2B illustrates a graph of a voltage to a time in a time-lapse electrophoretic voltage application method.

FIG. 3 schematically illustrates a view of a side surface of a biological sample staining apparatus according to an embodiment.

FIG. 4 schematically illustrates a schematic view of a biological sample staining apparatus using an electric field and magnetic focusing according to another embodiment.

FIG. 5 illustrates an exploded perspective view of a sample chamber of a biological sample staining apparatus according to an embodiment.

FIG. 6 illustrates an exploded perspective view of a sample chamber of a biological sample staining apparatus according to another embodiment.

FIG. 7 illustrates a perspective view of a biological sample staining apparatus according to an embodiment, and illustrates a state before inserting a sample chamber into an external chamber that is a biological sample fixing part.

FIG. 8 illustrates a partially enlarged perspective view of a biological sample staining apparatus according to an embodiment, and illustrates a state in which a biological sample holder is inserted into a biological sample fixing part.

FIG. 9 illustrates a perspective view of an appearance of a sample chamber of a biological sample staining apparatus according to an embodiment.

FIG. 10 illustrates a perspective view of an outer buffer circulation device of a biological sample staining apparatus according to an embodiment.

FIG. 11 illustrates a fluorescence image of a result obtained by staining a clarity sample of a brain tissue of an untransformed rat as a biological sample with an antibody through an electrophoretic method using an ion conductive film (scale bar: 100 μm).

FIG. 12 illustrates a fluorescence image of a result obtained by staining a clarity sample of a brain tissue of an untransformed rat as a biological sample with lectin through an electrophoretic method using an ion conductive film (scale bar: 100 μm).

### MODE FOR INVENTION

In order to describe the present invention, operational advantages of the present invention and an object achieved by an embodiment of the present invention, a preferred embodiment of the present invention will be illustrated, and the present invention will be described with reference to the embodiment. First, the terms used in the present application are used to describe only specific embodiments and are not intended to limit the scope of the present invention, and singular forms are intended to include plural forms unless the context clearly indicates otherwise. In addition, in the present specification, it should be understood that the term “include”, “comprise”, “have”, or “configure” indicates that a feature, a number, a step, an operation, a constituent element, a part, or a combination thereof described in the specification is present, but does not exclude a possibility of presence or addition of one or more other features, numbers, steps, operations, constituent elements, parts, or combinations, in advance. Further, in the present specification, a numerical range expressed as “A to B” means all values (real numbers) between A and B including A and B, and also includes approximate values of A and B recognized as an equal range.

An embodiment of the present invention provides a method for staining a biological sample, including the following steps, in staining a biological sample using an electric field:

(a) positioning a biological sample to be adjacent to a staining reagent for a biological sample, and separating the biological sample and the staining reagent for the biological sample from an outer buffer by using an ion conductive film; and

(b) forming an electric field so that a current flows through the ion conductive film to the staining reagent for the biological sample and the biological sample.

In the staining of the biological sample using the electric field, the staining reagent for the biological sample with a charge moves in the electric field and contacts the biological sample to stain a target biological material of the biological sample. By forcibly moving the staining reagent for the biological sample through the electric field, penetration efficiency and speed of the staining reagent into the sample are superior compared to passive staining of the biological sample that depends on diffusion.

The step (b) has the advantage of controlling the movement of the staining reagent for the biological sample by flowing a current through the ion conductive film, while preventing the denaturation of the staining reagent for the biological sample due to direct contact between the electrode and the staining reagent for the biological sample. More specifically, in the step (b) of forming the electric field (applying a voltage) so that the current passes through the ion conductive film and flows in the staining reagent for the biological sample and in the biological sample, as a frame that may physically separate the electrode, the staining reagent for the biological sample, and the biological sample from the external buffer, the ion conductive film may be used. Therefore, it is possible to secure fluidity of the frame and increase applicability and light accessibility. In addition, since the ion conductive film allows a current to pass through it, while the ion conductive film does not allow a biological sample to pass through it, it may block direct contact between the staining reagent for the biological sample and the electrode. Thus, it is possible to prevent the staining reagent for the biological sample from being denatured and simultaneously prevent external loss of the staining reagent for the biological sample.

More specifically, the step (b) may include a step of applying a voltage so that the current sequentially flows in an electrode (first electrode) of the same polarity as the staining reagent for the biological sample, the ion conductive film, the staining reagent for the biological sample, the biological sample, and an electrode (second electrode) of opposite polarity to the staining reagent for the biological sample (for example, in a forward and/or reverse direction).

The ion conductive film applied to the biological sample staining method of the present embodiment has very low electrical resistance, and thus it has very high electrical conductivity. In addition, since the ion conductive film does not include physical pores, it may prevent leakage of all organic molecules as well as macromolecules (for example, antibodies). Furthermore, the ion conductive film has good durability, and thus, may be used for a long time, and it is formed as a flat film, so that when it is implemented as a sample chamber, there are no restrictions on thickness, height, shape, and the like. Since the ion conductive film may pass only ions through a molecular channel formed between bonding structures of constituent molecules, a cation selective permeation electrolyte membrane, an anion selective permeation electrolyte membrane, and a cation/anion exchange membrane may be variously selected and applied according to experimental methods.

Meanwhile, in the biological sample staining method according to the embodiment, during electrophoresis using a cation selective permeation electrolyte membrane as an ion conductive film, H⁺ generated from the electrode of the external chamber flows into the sample chamber, so that the pH in the sample chamber may decrease with time.

That is, during electrophoresis using the cation selective permeation electrolyte membrane, H⁺ and OH⁻ ions from the electrode are generated by electrolysis of water, and at this time, the H⁺, which is a cation, passes through the cation selective permeation electrolyte membrane together with Li⁺, which is one of the buffer composition materials. Generally, acidity of the buffer used for processing the biological sample is about pH 9, and according to the electrophoresis, H⁺ generated from the electrode of the external chamber flows into the sample chamber, so that the pH in the sample chamber may decrease with time.

To prevent this pH drop, the following method may be used.

An appropriate distance (for example, about 20 mm or more) is maintained between the electrode and the ion conductive film, and perfusion may be performed at a predetermined flow rate or higher in a space between the electrode and the film. For example, when the applied voltage is 50 V, the appropriate distance between the electrode and the ion conductive film is approximately 20 mm or more, and the flow rate may be set to 500 ml/min or more to perform perfusion. As a result, hydrogen ions generated from the electrode may diffuse and be discharged to the external buffer container before reaching the electrolyte membrane, and the discharged buffer may be neutralized by mixing with a buffer containing OH formed in the opposite electrode.

During long-term electrophoresis, the pH drop may occur at a slow speed, and to prevent this, a composition of the buffer in the sample chamber and a composition of the buffer in the external chamber may be differently set. That is, the sample chamber may contain an electrolyte composition material lithium hydroxide (LiOH) or NaOH and a boric acid, which is appropriate for the sample at a pH of 9±0.5, while the external chamber may contain an electrolyte composition material lithium hydroxide (LiOH) or NaOH and a boric acid, which is appropriate for the sample at a pH of 10.6±0.5, which is higher than that of the sample chamber.

In addition to the electrolyte, the external chamber may further contain a pH buffering material such as Tris (tris(hydroxymethyl)aminomethane), MOPS, and the like having a concentration of 500 mM or less, and in addition to the electrolyte, the sample chamber may further contain a pH buffering material such as Tris, MOPS, and the like having a concentration of 100 mM or less.

Therefore, when the method is applied, the buffer pH in the sample chamber may be maintained at about 9 for more than 24 hours, which may be sufficient time for staining a biological sample having a diameter of 10 mm³, which is a size of the largest biological sample generally used.

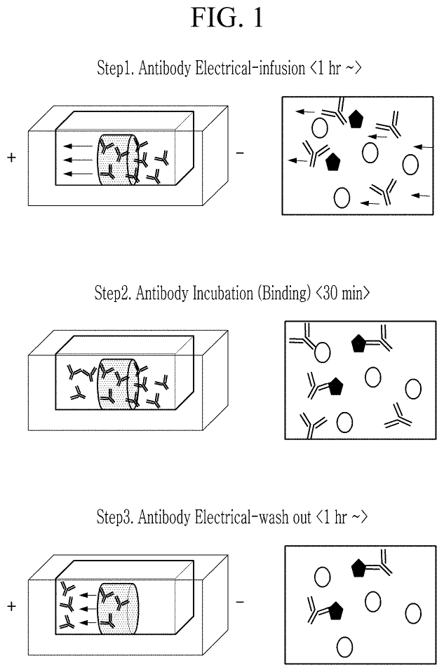

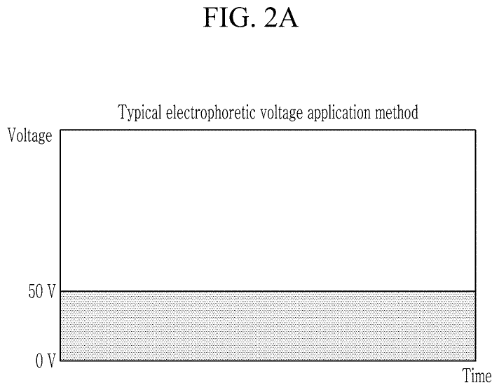

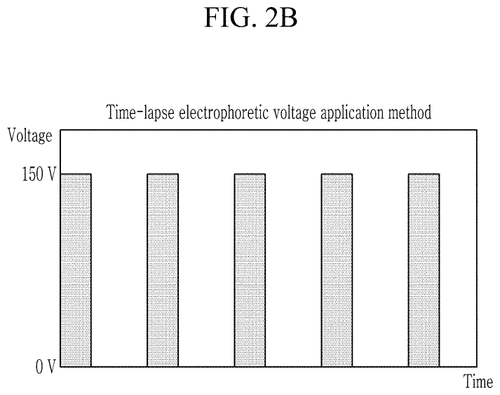

FIG. 1 illustrates a schematic view of a process of staining a biological sample using an electrophoretic method according to an embodiment, FIG. 2A illustrates a graph of a voltage to a time in a typical electrophoretic voltage application method, and FIG. 2B illustrates a graph of a voltage to a time in a time-lapse electrophoretic voltage application method.

In the biological sample staining method, in the step of forming an electric field, the staining reagent for the biological sample is introduced into the biological sample by providing power to move (infuse) the staining reagent for the biological sample into the biological sample by the electric field, and in this case, a voltage is applied so that a current of about 60 to about 100 mA, about 70 to about 90 mA, or about 75 to about 85 mA flows for about 1 to about 5 hours, about 1 to about 3 hours, or about 1 to about 2 hours (see step 1 in FIG. 1). In this case, the step of forming the electric field may be performed while changing a direction of the current at intervals of about 5 to about 60 minutes, about 5 to about 15 minutes, or about 8 to about 12 minutes. As such, by applying a voltage while changing the current direction at predetermined time intervals, a staining reagent for the biological sample that has passed through the biological sample in an unreacted state may be returned to its original position (to the same electrode side). Therefore, since the staining reagent for the unreacted biological sample may be reused, waste of the staining reagent for the biological sample may be prevented and the total amount thereof that is used may be reduced, and the staining efficiency may be further improved by repeatedly contacting the staining reagent for the biological sample with the biological sample.

In order to sufficiently react (bind) the staining reagent for the biological sample infused into the biological sample with a target biological material, after the forming step of the electric field, the biological sample staining method may further include reacting the staining reagent for the biological sample by leaving the reaction system for about 10 minutes to about 2 hours or about 10 minutes to about 1 hour (without applying a voltage) (see step 2 of FIG. 1).

That is, the general electrophoretic voltage application method is to equally and continuously supply a voltage as shown in FIG. 2A, while the voltage application method in the present embodiment may apply a time-lapse electrical control method as shown in FIG. 2B. The time-lapse electrical control method is a technique that applies a high voltage in a short period of time, then has an idle period, and then repeats the same pattern, enabling faster antibody inflow to a highly dense sample. In this case, total amounts of electric actually applied to the biological samples are the same, and it is possible to secure a time for discharging heat generated through the idle period.

In addition, the biological sample staining method may further include a washing step (see step 3 of FIG. 1) for removing the unreacted staining reagent for the biological sample after the forming step of the electric field and/or the reacting step of the biological sample staining reagent. The washing step may be performed for about 1 to about 3 hours or about 1 to about 2 hours.

The biological sample staining method including all of the forming step of the electric field, the leaving step, and the washing step may complete the staining of the biological sample within about 10 hours, within about 9 hours, within about 8 hours, within about 7 hours, within about 6 hours, or within about 5 hours (and at least about 2 hours or 2.5 hours).

On the other hand, another embodiment of the present invention may be utilized as an analysis method including a step of measuring a signal generated by the staining of the biological sample together with the staining step of the biological sample. The staining step of the biological sample is the same as that in the biological sample staining method previously described, and may further include a step of reacting the staining reagent for the biological sample and the biological sample by allowing them to stand without applying an electric field, and/or a washing step for removing and/or recovering the unreacted staining reagent for the biological sample.

The measuring step of the signal is performed by measuring a fluorescence signal and/or light emitting signal generated according to the used staining reagent for the biological sample by using an appropriate measuring part. The measuring may include collecting a signal, visualizing a signal, and/or digitizing (quantifying) signal strength and/or signal area (signal portion). The measuring part may be selected from all parts that may visualize and/or quantify the fluorescent signal and/or the light emitting signal, and for example, may be one or more of all kinds of commonly used fluorescent microscopes (for example, optical microscopes, laser microscopes, etc.), a light emitting measurement device, a fluorescent camera, and a light emitting signal digitizing (quantifying) device.

An analysis method using the biological sample staining may be all methods that visualize or quantify a biological material (for example, a protein, etc.) that is a target of a staining reagent for a biological sample, and for example, it may be selected from all methods of visualizing or quantifying the presence or absence of a target biological material in a tissue, a three-dimensional distribution pattern and/or a three-dimensional distribution position thereof, and/or a content thereof in a tissue.

In the biological sample staining method, the forming step of the electric field may be performed by a step of applying voltages to both electrodes. As described above, the temperature of the reaction system increases due to thermal energy generated by applying a voltage to both electrodes, and thus, a problem that the staining reagent for the biological sample (for example, a protein reagent such as an antibody) is denatured may occur. In order to solve this problem, the biological sample staining method may further include a step of cooling the reaction system in which the biological sample staining or the biological sample analysis is performed.

The cooling step may be performed by exchanging the electrode buffer and/or cooling the outside of the ion conductive film and/or the electrode part and/or the buffer supplier of the electrode part. In the embodiment, the cooling step may include a step of exchanging the electrode buffer, and/or a step of circulating cooling water in the outside of the biological sample holder blocked from the outside by the ion conductive film (for example, the side of the biological sample holder (for example, a pair of opposite sides in which the electrode is not positioned), the lower surface (bottom), and/or the upper surface), and/or in the outside and/or inside of the supplier of the buffer solution supplied to the electrode part. The cooling step may be continuously or intermittently performed so that the temperature of the reaction system is maintained at a temperature at which the staining reagent for the biological sample is not denatured. In the embodiment, when a protein such as an antibody is used as a staining reagent for a biological sample, the temperature at which the staining reagent for the biological sample is not denatured may be a temperature at which the protein is not denatured, for example, 37° C. or less, 35° C. or less, 30° C. or less, 25° C. or less, 20° C. or less, 15° C. or less, 10° C. or less, or 5° C. or less (a lower limit value of the temperature range is a freezing point or more of the buffer and/or cooling water used for cooling). The cooling step is performed by the circulating of the buffer and/or cooling water, thus the temperature of the buffer and/or cooling water used in this case may be adjusted within the above range by adjusting and circulating the above staining reagent for the biological sample in a temperature range at which it is not denatured.

In addition to the temperature condition, the biological sample staining method may be performed under conventional reaction conditions in which the staining reagent for the biological sample and the biological sample are not denatured or damaged (for example, a pressure (for example, a normal pressure range), a pH (for example, a neutral range (pH 6 to 8), etc.).

The biological sample staining method minimizes or does not cause the denaturation of the staining reagent for the biological sample through the use of the ion conductive film and/or through the maintaining of the reaction temperature, so the unreacted staining reagent for the biological sample that does not react with the target biological material may be recovered and reused. Therefore, the biological sample staining method of the biological sample may further include a step of recovering the unreacted staining reagent for the biological sample after completion of the reaction, and before, at the same time, and/or after the recovering step, it may further include a step of arbitrarily washing the biological sample. In the embodiment, the step of washing the biological sample may be performed by changing a current direction and flowing the current for 10 minutes to 2 hours, or 30 minutes to 90 minutes, but is not limited thereto.

According to the above-described biological sample staining method, it is possible to effectively stain and/or analyze even the inside of a thicker biological sample (for example, a biological tissue having a thickness of 0.5 mm or more, 0.75 mm or more, 1 mm or more, 1.25 mm or more, 1.5 mm or more, 1.75 mm or more, or 2 mm or more (an upper limit value may be a thickness of an organ to which the biological tissue is included, or 10 mm, 7.5 mm, 5 mm, 4 mm, 3 mm, or 2.5 mm)) than the conventional method, and it is possible to significantly reduce an amount of a staining reagents for a biological sample to be used (for example, it is possible to stain a biological tissue having a thickness of about 1 mm to about 3 mm or about 1.5 mm to about 2.5 mm (for example, about 2 mm) and a diameter of about 5 mm to about 10 mm with a staining reagent (e.g., an antibody) of about 1 to 2 μl (for example, CLARITY brain tissue, etc.).

In addition, according to the biological sample staining method, it is possible to significantly reduce a staining time of a biological sample due to high staining efficiency of a biological sample. In the embodiment, in the case of the biological sample staining method, staining of a biological sample can be completed within about 10 hours, within about 9 hours, within about 8 hours, within about 7 hours, within about 6 hours, or within about 5 hours, including a time (about 1 to about 5 hours, about 1 to about 3 hours, or about 1 to about 2 hours) of infusing a staining reagent for a biological sample into a biological sample, a reaction time (reaction (binding) time between a staining reagent for a biological sample and a target biological material; about 10 minutes to about 2 hours or about 10 minutes to about 1 hour), and a washing time (about 1 to about 3 hours or about 1 to about 2 hours) (it takes least about 2 hours or 2.5 hours) (see FIG. 1). This is significantly shortened compared to the conventional passive staining of a biological sample by diffusion, which takes at least 90 hours or more, for example, 96 hours to 120 hours, to complete biological sample staining.

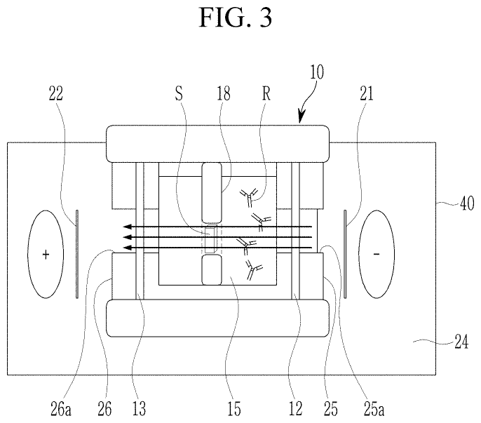

FIG. 3 schematically illustrates a view of a of side surface of a biological sample staining apparatus (reaction system) according to an embodiment.

In the biological sample staining method, the above-described steps may be performed in a reaction system that includes ion conductive films 12 and 13; a staining reagent for a biological sample R included in a staining reagent 15 for a biological sample blocked from a conductive medium 24 by the ion conductive films 12 and 13; a biological sample S fixed in a biological sample holder 18; and a first electrode 21 and a second electrode 22 positioned on sides of a pair of the ion conductive films 12 and 13 facing each other, as shown in FIG. 3. The reaction system may be filled with the conductive medium 24 (for example, a conventional buffer solution). The biological sample S may be immobilized in a sample chamber 10 so that a wide cross-section thereof faces both electrodes 21 and 22 (that is, in a direction parallel to the side surfaces of the ion conductive films 12 and 13 where both electrodes 21 and 22 are positioned), and the staining reagent R for the biological sample may be supplied with power to move to the biological sample S and to penetrate into the sample by an electric field.

In the present embodiment, the ion conductive films 12 and 13 may be cation selective permeation electrolyte membranes. The cation selective permeation electrolyte membranes allow a current to flow by selectively permeating only cations (for example, lithium ions when using a LiOH buffer, and sodium ions when using a NaOH buffer) among electrolytes in the buffer during electrophoresis. The cation selective permeation electrolyte membrane does not leak out only macromolecules (antibodies) in the sample chamber 10, but also the buffer compositions to an external chamber 40 (chamber where the electrode is positioned), thus the antibody concentration and buffer concentration in the sample chamber 10 may be perfectly maintained. This feature may prevent leakage of Tris (tris(hydroxymethyl)aminomethane) used for pH buffering, thereby enabling stable acidity maintenance. When the cation selective permeation electrolyte membrane is immersed in the buffer and wetted with water, the electrical resistance may be extremely low. For this reason, it is only necessary to apply a voltage to be applied in the actual sample chamber 10 to an electrode of the external chamber. That is, since an amount of electricity applied to the actual sample chamber 10 may be the same as an amount of electricity applied to the external chamber 40, and since the applied amount of electricity may be accurately known without electrical loss by the sample chamber 10, reproducibility of an experiment and stability of a sample may be secured.

In contrast, in the case of using a nano-pore membrane as a comparative example, the electrical resistance is very large, and a voltage that is much higher than a voltage to be applied in the actual sample chamber 10, for example tens to hundreds of percent higher, must be inverted or inferred to be used, and as a result, electrical stability is poor and a lot of heat is generated, and an amount of electricity in the actual sample chamber 10 may not be accurately measured, thus a sample may be damaged and a staining rate may decrease.

Meanwhile, the first electrode 21 and the second electrode 22 may be configured of an electrode part including the conductive medium 24 (for example, a conventional buffer solution) outside the side surfaces of the ion conductive films 12 and 13. The electrode part includes the first electrode 21 having the same polarity as the electric charge of the staining reagent part for the biological sample at the outside of the side of the ion conductive film 12 and 13 at the side of the staining reagent part 15 for the biological sample, and the second electrode 22 having a polarity opposite to that of the staining reagent for the biological sample at the outside of the side of the buffer part 16 at the opposite side thereof (for example, when a negatively charged material such as an antibody is used as a staining reagent for a biological sample, a cathode is formed at the side of the staining reagent for the biological sample, and an anode is formed at the opposite side).

To prevent denaturation of the staining reagent (for example, proteins such as antibodies) for the biological sample by heat generated from the electrodes 21 and 22, a cooling water circulation channel (not shown) may be additionally included outside the sample chamber 10. The cooling water circulation channel may be positioned on a side surface, a bottom surface, and/or an upper surface except a pair of side surfaces where the electrodes 21 and 22 are positioned, and the sample chamber 10 and the cooling water circulation channel are positioned in contact with each other at an interval of about 0 to 0.5 mm or less so that there is no loss of the electric field, but is not limited thereto.

A shape of the sample chamber 10 is not particularly limited, and may be a rectangular parallelepiped shape with an empty space inside (a rectangular parallelepiped shape with an open surface (upper surface) parallel to a major axis) for convenience in use and/or in manufacturing, but is not limited thereto. When the sample chamber 10 has a rectangular parallelepiped shape with one surface (upper surface) open, the electrode part may be positioned at both end surfaces of the major axis of the rectangular parallelepiped shape, the cooling water circulation channel may be positioned at both side surfaces and/or the bottom surface parallel to the major axis, and optionally, after the biological sample holder 18 is fixed to the biological sample fixing part, the cooling water circulation channel may be covered on the upper surface of the sample chamber 10.

The biological sample staining apparatus may further include a buffer supplier and/or a cooling water supplier. The buffer supplier circulates the buffer of the electrode part to prevent a temperature rise due to heat generated from the electrode. For this purpose, the buffer supplier may be connected to the electrode part to supply a temperature-controlled buffer.

In another embodiment, the biological sample staining apparatus further includes a visualization and/or quantification device for a signal (for example, a fluorescence signal) generated by the reaction between the staining reagent for the biological sample and the biological sample for real-time analysis (real-time monitoring). The visualization and/or quantification device for the signal may be one or more selected from a group consisting of a light source, a lens, an imaging device, and an operation device, for example, one or more selected from a group consisting of a fluorescence microscope (for example, an optical microscope or a laser microscope), a fluorescent camera, a display (monitor), and a computer, but is not limited thereto.

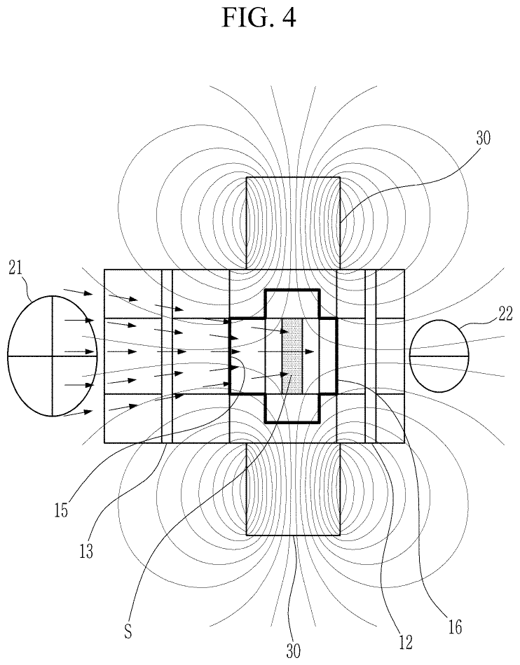

FIG. 4 schematically illustrates a view of a biological sample staining apparatus using an electric field and magnetic focusing according to another embodiment.

Referring to FIG. 4, in the biological sample staining apparatus according to the present embodiment, the electrodes 21 and 22 are disposed at the front and rear of the sample chamber 10, and magnetic bodies 30 are disposed at the left and right thereof. Here, a front-rear direction of the sample chamber 10 may be defined as a first direction parallel to an opposite direction of the electrodes 21 and 22 of different polarities, and a left and right direction may be defined as a second direction orthogonal to the front-rear direction. In the biological sample staining apparatus according to the present embodiment, the first electrode 21, the staining reagent part 15 for the biological sample, the biological sample S, the buffer part 16, and the second electrode 22 are arranged in the first direction, and the magnetic body 30 is disposed at the left and right sides of the sample chamber 10 to be adjacent to the biological sample S. As described above, the sample chamber 10 may be formed of a non-conductive structure, and may include the ion conductive films 12 and 13 that are disposed to face the first electrode 21 and the second electrode 22, respectively.

In the present embodiment, the magnetic body 30 is positioned at the left and right thereof to be adjacent to the biological sample S, thereby forming a magnetic field during the process of the staining reaction to induce magnetic focusing. By using the electric field and magnetic focusing together as described above, it is possible to prevent the spreading of the staining reagent for the biological sample to parts other than the sample, and simultaneously, to increase the penetration efficiency of the staining reagent for the biological sample.

Respective magnetic bodies 30 positioned at the left and right sides of the sample chamber 10 to be adjacent to the biological sample S may be positioned to be parallel to each other along the first direction. In addition, as another example, respective magnetic bodies 30 positioned at the left and right sides of the sample chamber 10 may be disposed to be inclined toward each other by a predetermined angle with respect to the first direction. For example, when the left magnetic body 30 based on the first direction of respective magnetic bodies 30 at the left and right sides is inclined by 15° in the clockwise direction and the right magnetic body 30 thereof is inclined by 15° in the first direction, the magnetic focusing effect may be further improved. However, the present invention is not limited to the angle.

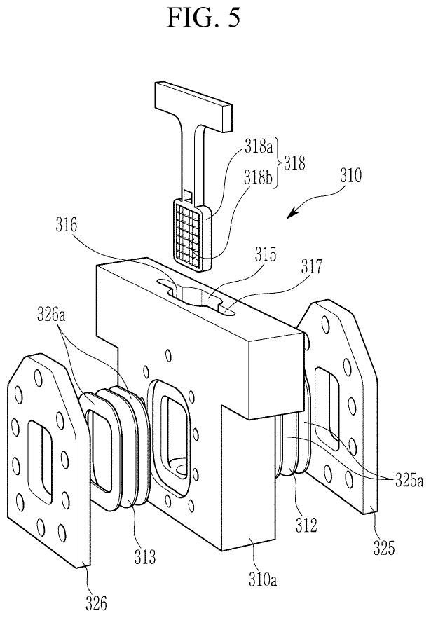

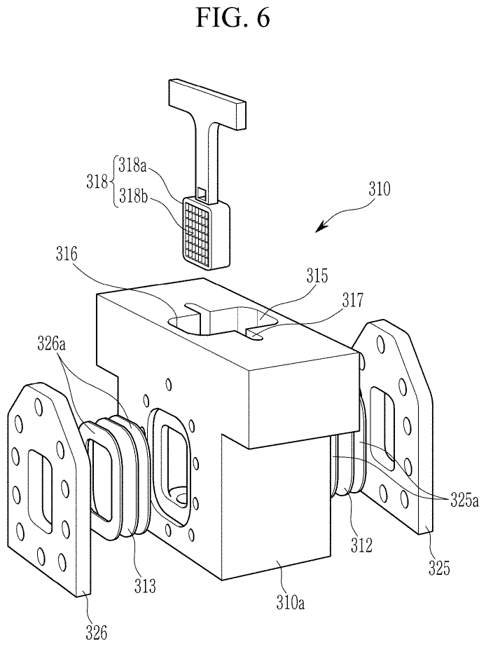

FIG. 5 illustrates an exploded perspective view of a sample chamber of a biological sample staining apparatus according to an embodiment, and FIG. 6 illustrates an exploded perspective view of a sample chamber of a biological sample staining apparatus according to another embodiment.

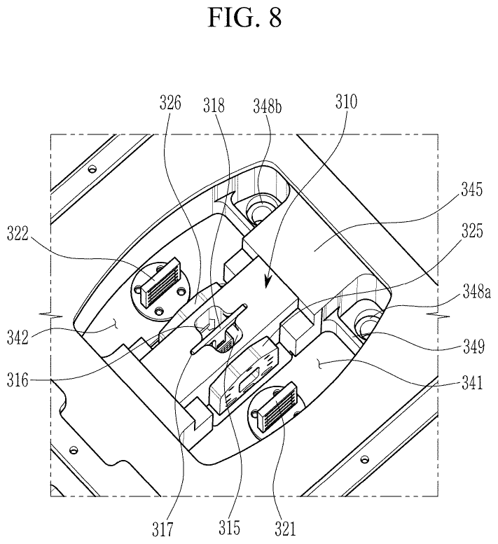

Referring to FIG. 5 and FIG. 6, a sample chamber 310 of the biological sample staining apparatus according to the present embodiment includes a sample chamber frame 310a in which a biological sample holder 318 is inserted and fixed, and ion conductive films 312 and 313 are disposed at front and rear sides of the sample chamber frame 310a. Here, a front-rear direction of the sample chamber 310a may be defined as a first direction parallel to an opposite direction of electrodes 321 and 322 (see FIG. 8) of different polarities, and a left and right direction may be defined as a second direction orthogonal to the front-rear direction.

The sample chamber frame 310a of the sample chamber 310 is made of a non-conductive structure having an inner space, and the sample chamber frame 310a may have an open upper surface. The space formed inside the sample chamber frame 310a is an empty space, and a staining reagent part 315 for a biological sample, a buffer part 316, and a biological sample fixing part 317 may be included therein. The biological sample fixing part 317 is positioned between the staining reagent part 315 and the buffer part 316 for the biological sample.

The staining reagent part 315 for the biological sample is a space in which a staining reagent is included, and the buffer part 316 is a space in which a buffer solution is included. The staining reagent part 315 for the biological sample may have a space (volume) of a size capable of carrying a staining reagent for a biological sample required to sufficiently stain a biological sample to be loaded. The buffer part 316 is a space to be filled with a buffer solution, and is a hole (mesh position) of the biological sample holder 318 or a space in which the staining reagent for the biological sample that has passed through the biological sample loaded therein is collected.

The biological sample fixing part 317 is an inner space of the sample chamber frame 310a in which the biological sample holder 318 carrying the biological sample is fixed (inserted). The biological sample holder 318 includes a holder body 318a having a hole therein and a mesh 318b positioned at both sides of the hole to cover the hole. The holder body 318a has an approximate T-shape such that a user may easily insert and fix the biological sample in the sample chamber frame 310a. In order to prevent the antibodies from sinking during electrophoresis, a magnetic spin bar may be placed at the bottom of the sample chamber frame 310a, and the antibodies may be evenly mixed by turning a stirrer outside the sample chamber frame 310a.

The biological sample is loaded into the space between the hole in the holder body 318a and the mesh 318b at both sides thereof. In order to load a biological sample, the mesh 318b positioned at both sides may have all or a portion (for example, ½ or more or ¾ or more of the circumference) of its circumference detachable to the holder body 318a. For example, all of a circumference of one of the meshes 318b positioned on both surfaces of the hole of the holder body 318a may be attached to the holder body 318a to form one surface on which a biological sample may be loaded, and after placing the biological sample thereon, the mesh 318b on the opposite surface is covered and a portion or all of the circumference is attached to the holder body 318a to be able to load the biological sample between the meshes 318b on both surfaces. In this case, the mesh 318b is positioned on both surfaces contacting a cross-section of a large area of the supported biological sample, and has pores through which the staining reagents (for example, antibodies) for the biological sample may pass.

A thickness and hole size of the holder body 318a may be determined according to a size of the biological sample being loaded, for example, 1 to 1.5 times, 1 to 1.4 times, 1 to 1.3 times, 1 to 1.2 times, 1 to 1.1 times, or 1 to 1.05 times an average thickness of the biological sample to be loaded and/or an average diameter of a wide cross-section thereof.

The biological sample holder 318 is fixed (inserted) inside the biological sample fixing part 317 so that the cross-section of the large area of the biological sample loaded therein is positioned in a direction parallel to the long axis of the biological sample fixing part 317 in the sample chamber frame 310a. The biological sample fixing part 317 has a width into which the biological sample holder 318 may be inserted, for example, 1 to 1.5 times, 1 to 1.4 times, 1 to 1.3 times, 1.2 times, 1 to 1.1 times, or 1 to 1.05 times an average thickness of the biological sample.

The biological sample fixing part 317, in order to stably fix the biological sample holder 318 and to separate (block) the staining reagent part 315 and the buffer part 316 for the biological sample, may have a space in which grooves are formed in the inner walls of a pair of facing side surfaces of the sample chamber frame 310a where both ends of the biological sample fixing part 317 are positioned such that the inner wall of the sample chamber frame 310a extends toward the outer wall thereof.

The sample chamber shown in FIG. 6 has a thicker sample chamber frame than the sample chamber shown in FIG. 5, and thus includes a staining reagent part and a buffer part for a biological sample having a larger space, and a biological sample fixing part. Therefore, a thicker biological sample holder may be mounted on the biological sample fixing part shown in FIG. 6. Accordingly, it is possible to stain samples of various sizes by changing only the size of the sample chamber frame.

The holder body 318 may made of a material through which a staining reagent for electrical and biological samples, and if necessary, a buffer, do not pass. Therefore, the staining reagent for the biological sample, which moves by the formation of an electric field, may only move through the mesh 318b positioned in the hole of the holder body 318a when passing through the biological sample holder 318, so it may be more concentrated on the biological sample loaded between the meshes 318b.

Meanwhile, film fixing plates 325 and 326 that may press and fix the ion conductive films 312 and 313 may be positioned outside a pair of side surfaces facing each other in the first direction of the sample chamber frame 310a, that is, in the front-rear direction. That is, the inner space of the sample chamber frame 310a is open in the front-rear direction, and by covering it with the ion conductive films 312 and 313, the biological sample fixing part 317 may be separated (blocked) from the staining reagent part 315 and the buffer part 316 for the biological sample. In this case, the film fixing plates 325 and 326 are coupled to the sample chamber frame 310a by using a plurality of screws, and function to fix the ion conductive films 312 and 313 in close contact with the sample chamber frame 310a, and are fixed ion conductive films, and in order to secure the sealing properties of the fixed ion conductive films 312 and 313, gaskets 325a and 326a for film sealing are interposed in the front and rear sides of respective ion conductive films 312 and 313 to be fixed together with the ion conductive films. These film fixing plates 325 and 326 and gaskets 325a and 326a for film sealing may have openings corresponding to the inner space formed in the sample chamber frame 310a.

Since the ion conductive films 312 and 313 have a property that they flexibly stretch when contacting water, the gaskets 325a and 326a for film sealing are double-padded to respective ion conductive films 312 and 313 to be coupled to each other by using a plurality of screws, and thus sufficient sealing effect may be obtained even in water.

In the above, the sample chamber 310 may be defined as including the biological sample holder 318, and in this case, the biological sample holder 318 may include the holder body 318a containing or not containing a biological sample.

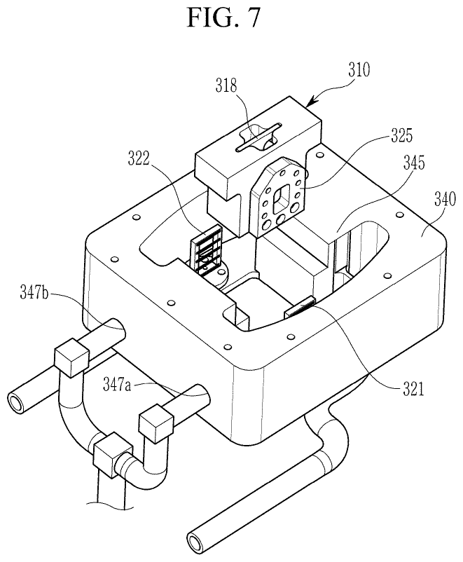

FIG. 7 illustrates a perspective view of a biological sample staining apparatus according to an embodiment, and illustrates a state before inserting a sample chamber into an external chamber that is a biological sample fixing part, and FIG. 8 illustrates a partially enlarged perspective view of a biological sample staining apparatus according to an embodiment, and illustrates a state in which a biological sample holder is inserted into a biological sample fixing part.

Referring to FIG. 7, the biological sample staining apparatus 300 according to the present embodiment includes an outer chamber 340, a sample chamber 310 fixed thereto, and electrodes 321 and 322 fixed in the outer chamber 340. Here, a front-rear direction of the sample chamber 310 may be defined as a first direction parallel to an opposite direction of the electrodes 321 and 322 of different polarities, and a left and right direction may be defined as a second direction orthogonal to the front-rear direction.

Referring to FIG. 8, the outer chamber 340 of the biological sample staining apparatus may be largely partitioned into two spaces by a horizontal wall 345 to be separated into a first space 341 and a second space 342. The first electrode 321 is positioned in the first space 341, and the second electrode 322 is positioned in the second space 342. A middle portion of the horizontal wall 345 forms an open portion partially opened so that the first space 341 and the second space 342 communicate with each other, and a width of the open portion is opened as much as the size corresponding to that of the sample chamber 310. Therefore, when the sample chamber 310 is inserted into the open portion of the horizontal wall 345, the first space 341 and the second space 342 may be disconnected from each other.

The outer chamber 340 includes two buffer inlets 347a and 347b and two buffer outlets 348a and 348b. The buffer inlets include a first inlet 347a connected to the first space 341 and a second inlet 347b connected to the second space 342, and respective inlets 347a and 347b are positioned at a lower end portion of the outer chamber 340 to supply a cooling buffer into respective spaces 341 and 342. The buffer outlets 348a and 348b include a first outlet 348a through the first space 341 and a second outlet 348b through to the outside from the second space 342. Each of the outlets 348a and 348b is opened upward at the upper end portion of each of the spaces 341 and 342 of the outer chamber 340. Therefore, the cooling buffer flowing into the buffer inlets 347a and 347b almost fills each of the spaces of the outer chamber 340 and then overflows and flows out to each of the buffer outlets 348a and 348b. In addition, a buffer level maintenance dam 349 is formed to protrude to be adjacent to each of the buffer outlets 348a and 348b, so that a certain level of buffer may be maintained in each of the spaces 341 and 342 of the outer chamber 340.

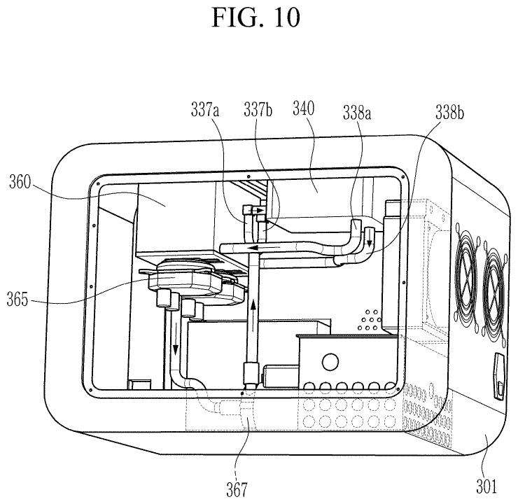

In this case, the sample chamber 310 may prevent the flow of fluid within the sample chamber 310 to protect the sample. The outer chamber 340 may have a structure in which fluid is rapidly circulated at the inside thereof to remove heat generated from the electrode, and it is possible to prevent a pH drop by rapidly circulating fluid so that H⁺ generated from the electrode does not flow into the sample chamber 310. Here, H⁺ flowing into the buffer supplier 360 (see FIG. 10) may be neutralized by mixing with OH⁻ generated from an opposite electrode.

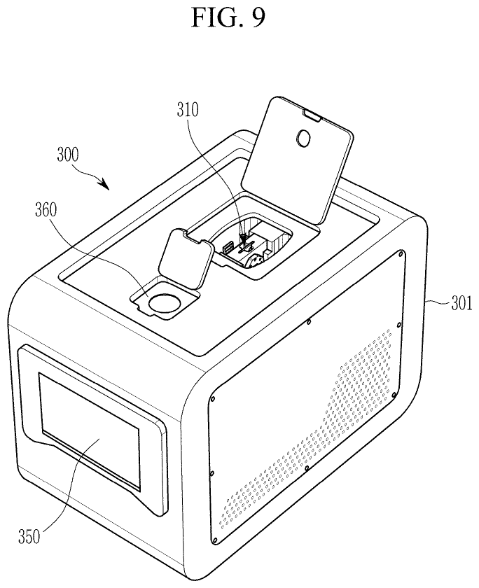

FIG. 9 illustrates a perspective view of an appearance of a sample chamber of a biological sample staining apparatus according to an embodiment, and FIG. 10 illustrates a perspective view of an outer buffer circulation device of a biological sample staining apparatus according to an embodiment.

Referring to FIG. 9, the biological sample staining apparatus 300 according to the present embodiment has a body 301 of a substantially rectangular parallelepiped shape in which some corners are rounded, and an outer chamber 340 in which the sample chamber 310 is fixed to an upper end portion thereof such that a biological sample staining process is performed. The buffer supplier 360 is positioned at an upper portion of the outer chamber 340, and a controller 350 exposed in a form of a display panel may be disposed on a front surface. Electrophoretic voltage and current control, buffer temperature control, periodic current direction control, and time-lapse electric control may be performed by the controller 350.

Referring to FIG. 10, inflow pipes 337a and 337b may be connected to the buffer inlets 347a and 347b of the outer chamber 340, and outflow pipes 338a and 338b may be connected to the buffer outlets 348a and 348b of the outer chamber 340. The outflow pipes 338a and 338b are connected to the buffer supplier 360 to recover the buffer solution discharged from the outer chamber 340, and may pass it through a cooling part 365 including a thermoelectric element and a water-cooled cooler to cool it. The buffer solution cooled while passing through the cooling part 365 may be pumped by a buffer circulation pump 367 to be supplied to the buffer inlets 347a and 347b of the outer chamber 340 through the inflow pipes 337a and 337b. The cooled buffer solution supplied in this way may serve to lower the reaction temperature of the sample chamber 310 (that is, it may serve to maintain the reaction temperature to be equal to or less than that of the staining reagent for the biological sample and/or the denaturation temperature of the biological sample).

Meanwhile, the biological sample described in the present specification may be cells isolated from vertebrates and invertebrates such as animals, such as insects, xenopus, zebrafish, mammals (for example horses, cattle, sheep, dogs, cats, murine, rodents, non-human primates, or humans), or a culture of the cell, tissue thereof, or organ thereof, but is not limited thereto. The biological sample may be collected (or separated) from a living subject (for example, a biopsy sample), or may be collected from a dead subject (for example, an autopsy or necropsy sample). The living subject may be selected from various tissues and organs, for example, hematopoietic, nerve (central or peripheral), glial, mesenchymal, skin, mucous membrane, interstitium, muscle (skeletal, heart, or smooth), spleen, reticular endothelium, epithelium, endothelium, liver, kidney, pancreas, gastrointestinal, lung, or fibroblast. As one example, the biological sample may be a brain tissue isolated from mammals, including vertebrates, for example humans, or forebrains of rodents, but is not limited thereto.

Since the biological sample separated from a living body contains various materials other than the biological material to be analyzed, it is an obstacle to obtaining an accurate analysis result, so the biological sample may be a sample in which a biological material, such as lipids which interfere with analysis (for example, optical analysis), other than the biological material (for example, protein and/or nucleic acid molecules), to be analyzed is removed.

The biological sample applicable to the present invention may be separated from the living body.

The present invention has the advantage of being applicable to a relatively thick biological sample, and in this respect, the biological sample may be a biological tissue having a thickness of 0.2 mm or more, 0.3 mm or more, 0.5 mm or more, 0.75 mm or more, 1 mm or more, 1.25 mm or more, 1.5 mm or more, 1.75 mm or more, or 2 mm or more (an upper limit value may be a thickness of an organ to which the biological tissue is included, or 10 mm, 7.5 mm, 5 mm, 4 mm, 3 mm, or 2.5 mm), but is not limited thereto, and the present invention may be applied to biological samples thinner than the above range. A cross-section of the biological sample may have a shape close to a circle having a diameter of about 5 mm to 10 mm, but is not limited thereto, and may be appropriately determined according to a size and/or shape of a holder body.

The staining reagent for the biological sample described in the present specification means that one or more selected from a group consisting of materials (for example, target binding proteins such as antibodies and lectins, aptamers, target binding nucleic acid molecules such as antisense RNA, siRNA, shRNA, and small molecular chemicals (for example, an organic compound having a chromophore that binds to a target biological material by electrostatic binding; for example, at least one selected from a group consisting of methylene blue, toluidine blue, hematoxylin, eosin, acid fuchsin, orange G, DAPI (4′,6-diamidino-2-phenylindole)) targeting a specific biological material (for example, protein, sugar, nucleic acid (DNA or RNA)) in a biological sample (for example, biological tissue)) are labeled as a detectable labeling material according to a typical method as necessary. As an example, the staining reagent for the biological sample may be charged.

A portion to be stained by the biological sample staining method provided in the present specification is not particularly limited, and may be one or more selected from a group consisting of a cell membrane, cytoplasm, nucleus, nuclear membrane, and various intracellular organelles, and it is possible to select a typical staining reagent for a biological sample suitable for a portion to be stained.

The labeling material may be one or more selected from all materials that generate a detectable signal (for example, fluorescence). For example, the fluorescent material may be one or more selected from a group consisting of the following materials, but is not limited thereto:

(1) Fluorescent protein: green fluorescent protein (GFP), yellow fluorescent protein (YFP), orange fluorescent protein (OFP), cyan fluorescent protein (CFP), blue fluorescent protein (BFP), red fluorescent protein (RFP), ultra red fluorescent protein, near-infrared fluorescent protein, etc.,

(2) Fluorescent protein variants: GFP variants such as Emerald (Invitrogen, Carlsbad, Calif.), EGFP (Clontech, Palo Alto, Cal), Azami-Green (MBL International, Woburn, Mass.), Kaede (MBL International, Woburn, Mass.), ZsGreenl (Clontech, Palo Alto, Calif.), CopGFP (Evrogen/Axxora, LLC, San Diego, Calif.), etc.; CFP variants such as Cerulean (Rizzo, Nat Biotechnol. 22(4):445-9 (2004)), mCFP (Wang et al., PNAS USA. 101(48):16745-9 (2004)), AmCyanl (Clontech, Palo Alto, Cal), MiCy (MBL International, Woburn, Mass.), CyPet (Nguyen and Daugherty, Nat Biotechnol. 23(3):355-60 (2005)); BFP variants such asEBFP (Clontech, Palo Alto, Calif.), etc.; YFP variants such as EYFP (Clontech, Palo Alto, Calif.), YPet (Nguyen and Daugherty, Nat Biotechnol. 23(3):355-60 (2005)), Venus (Nagai et al., Nat. Biotechnol. 20(1):87-90 (2002)), ZsYellow (Clontech, Palo Alto, Cal), mCitrine (Wang et al., PNAS USA. 101(48):16745-9 (2004)), etc.; OFP variants such as cOFP (Strategene, La Jolla, Calif.), mKO (MBL International, Woburn, Mass.), mOrange, etc.

(3) Non-protein organic fluorescent dyes: xanthene derivatives such as fluorescein, rhodamine, Oregon green, eosin, and Texas red; cyanine derivatives such as cyanine, indocarbocyanine, oxacarbocyanine, thiacarbocyanine, and merocyanine; squaraine derivatives such as Seta, SeTau, and Square dyes, and cyclosubstituted squaraines; naphthalene derivatives (dansyl and prodan derivatives); coumarin derivatives; oxadiazole derivatives such as pyridyloxazole, nitrobenzoxadiazole, and benzoxadiazole; anthracene derivatives such as anthraquinones, including DRAQS, DRAQ7, and CyTRAK Orange; pyrene derivatives such as cascade blue; oxazine derivatives such as Nile red, Nile blue, cresyl violet, and oxazine 170; acridine derivatives such as proflavin, acridine orange, acridine yellow, and the like; arylmethine derivatives such as auramine, crystal violet, and malachite green; tetrapyrrole derivatives such as porphin, phthalocyanine, bilirubin (for example, CF dye (Biotium), DRAQ and CyTRAK probes (BioStatus), BODIPY (Invitrogen), Alexa Fluor (Invitrogen), DyLight Fluor (Thermo Scientific, Pierce), Atto and Tracy (Sigma Aldrich), FluoProbes (Interchim), Abberior dyes (Abberior), DY and MegaStokes dyes (Dyomics), Sulfo Cy dyes (Cyandye), HiLyte Fluor (AnaSpec), Seta, SeTau, and Square Dyes (SETA BioMedicals), Quasar and Cal Fluor dyes (Biosearch Technologies), SureLight Dyes (APC, RPEPerCP, Phycobilisomes) (Columbia Biosciences), APC, APCXL, RPE, BPE (Phyco-Biotech, Greensea, Prozyme, Flogen), Vio Dyes (Miltenyi Biotec), etc.).

The ion conductive film described in the present specification may be applied by variously selecting a cation selective permeation electrolyte membrane, an anion selective permeation electrolyte membrane, and a cation-anion exchange membrane according to the experimental method, and only ions may pass through molecular channels formed between bonding structures of constituent molecules.

The ion conductive film described in the present specification has very low electrical resistance, and thus it has high electrical conductivity. In addition, since the ion conductive film does not include physical pores, it may prevent leakage of all organic molecules as well as macromolecules (for example, antibodies). Furthermore, the ion conductive film has good durability, and thus, may be used for a long time, and it is formed as a flat film, so that when it is implemented as a sample chamber, there are no restrictions on thickness, height, shape, and the like.

The sample chamber frame and the holder body used in the present specification may be made of a solid material that does not conduct electricity and does not pass the buffer and the immunostaining reagent. In order to not cause obstacles due to refraction, scattering, or dispersion of light during optical analysis, the sample chamber frame and the holder body may be made of a transparent material. For example, the chamber frame and the holder body may be made of the same or different materials, and each, independently, may be made of one or more selected from a group consisting of non-conductive materials such as acryl, glass, plastic, rubber, ceramics, and petroleum compounds. It may be made of the above material.

The cooling water circulation channel has a cooling water circulation passage connected inside, and may have any type of structure in which all surfaces except the cooling water inlet and outlet are closed and sealed, but there is no special limitation on the material, and any material that has excellent thermal conductivity and may circulate a liquid without loss is sufficient.

The mesh included in the holder body may be made of one or more of materials selected from a group consisting of silk, linen, and petroleum compound-derived fibers, but is not limited thereto. In addition, the mesh may have pores of a size through which the biological sample may not pass but the staining reagent for the biological sample may pass. For example, when an antibody is used as the staining reagent for the biological sample, it may have pores with an average diameter of about 30 nm or more, about 50 nm or more, about 70 nm or more, about 100 nm or more, or about 1 μm or more so that the antibody may pass through (a maximum value of the pore diameter may be equal to or less than the size through which the loaded biological sample may not pass and may be hold in the loading frame). In the embodiment, the mesh may have pores of an average diameter of 30 nm to 100 μm, 50 nm to 100 μm, 70 nm to 100 μm, 100 nm to 100 μm, 1 μum to 100 μm, 30 nm to 10 μm, 50 nm to 10 μm, 70 nm to 10 μm, 10 nm to 10 μm, or 1 μm to 10 μm, but is not limited thereto.

The inner space and the electrode part of the sample chamber may be filled with a typically used buffer solution. In the embodiment, the buffer solution may be selected from buffer solutions including an ionization providing material (electrolyte). The ionization providing material is not particularly limited, and for example, it may be one or more selected from a group consisting of lithium hydroxide, sodium chloride, potassium chloride, sodium hydroxide, etc., but is not limited thereto, and any material capable of ionization may be used. The buffer solution may be one or more selected from a group consisting of borate buffer, phosphate buffer saline (PBS), phosphate buffer, Tyrode buffer, tris buffer, glycine buffer, citrate buffer, and acetate buffer, but is not limited thereto. In the embodiment, the buffer solution may contain 50 mM of lithium hydroxide, but is not limited thereto.

On the other hand, as another embodiment of the present invention, it is possible to implement a method of clarifying a biological tissue by electrophoresis by using an ion conductive film and an electric field. That is, it can be implemented by applying a clarification reagent in the above-described biological sample staining method.

The reagent that has been typically used for clarifying the biological tissue is sodium dodecyl sulfate (SDS), and the SDS dissolves lipids, which are the main constituents of cell membranes of biological samples, to clarify the biological samples. The SDS clarifying method has been mainly used as a passive clearing method (in which a sample is immersed in an SDS solution and a lipid is dissolved and cleared according to natural diffusion). However, this method takes a lot of time for tissue clearing due to the low tissue penetration of the macromolecule SDS.

An SDS acceleration method by electrophoresis is used as a method to increase such low tissue penetration, but it has many problems such as oxidation of SDS by electrophoresis and contamination of the sample by a carbon oxide generated in the electrode. The recently announced nano-pore membrane-mediated electrophoresis technology may remove the carbon oxide, but due to its high electrical resistance, a very high electrical force must be used, and thus the pores of the membrane may be clogged due to sample damage or over-generated carbon oxide.

The electrophoretic biological clarifying method using an ion conductive film according to the present embodiment has almost no electrical resistance, thus uses only appropriate electrical force required for clarifying, and minimizes occurrence of the carbon oxide, thereby continuously maintaining performance of the ion conductive film. Since the carbon oxide may not pass through the ion conductive film, it is possible to achieve clear biological sample clarity without any contamination. In addition, unlike a nano-pore membrane, the ion conductive film does not have physical holes, so that clogging of holes by the carbon oxide or the like is fundamentally blocked. This property does not require exchanging the film even in a long-term clarifying experiment, so that efficient biological tissue clarity may be achieved.

Hereinafter, embodiments of the present invention will be described in more detail. The following embodiments are cases of performing an immunostaining method using an antibody for staining a biological sample. These embodiments are only for explaining the present invention in more detail, and that a scope of the present invention is not limited to these embodiments will be clearly understood by a person of ordinary skill in the art.

### Example 1: Preparation of Sample

A cleared brain tissue sample was prepared by a typical CLARITY method using the brain of an untransformed rat (SD Rat, 4-6 weeks old).

Through heart perfusion, blood was drained from the brain microvessels. The brain was excised from the rat, was immersed in a hydrogel monomer solution in which 4% (w/v) acrylamide, 0.25% (w/v) VA-044, and 4% (w/v) PFA (paraformaldehyde) was dissolved in a phosphate buffer saline (PBS) solution, and was incubated for 2 days at 4° C.

Then, the brain was placed in a vacuum state for 2 to 4 hours in the dark while raising a temperature to 37° C. by using a specially manufactured machine (CLARITY Easy-Imbedding, LCI).

Thereafter, after slicing it at a desired sample size (thickness: 500 μm, 1 mm, 1.5 mm. 2 mm, 5 mm; diameter: 5 mm, 10 mm), Electro-Tissue Clearing (ETC) was performed by using a CLARITY machine (CLAIRT Easy-Clearing, LCI). In this case, a buffer solution containing 4% of SDS, 50 mM of LiOH, and 25 mM of boric acid was used. The clearing was performed under a condition of 50 to 70 V and 35° C., and was performed for 1 to 5 days depending on the size of the sample.

The tissue that had completed the CLARITY process as described above was immersed in a borate buffer (50 mM LiOH, 25 mM of boric acid) for 1 day under a condition of 37° C. and washed to remove all residual SDS that interferes with antibody binding.

### Example 2: Preparation of Biological Sample Staining Apparatus Using Electrophoretic Method of Staining Biological Sample with Ion Conductive Film Applied

The manufactured sample chamber was installed between respective electrode parts of a device including a power supply, both electrode parts (each electrode part includes a buffer inlet at a lower portion of one side and a buffer outlet at an upper portion of the opposite side), a buffer supply part (borate buffer; 50 mM of LiOH, 25 mM of boric acid) connected to a buffer inlet and a buffer outlet of the both electrode parts, and a cooler connected to the buffer supply part. The device includes a cooling water circulation channel that is disposed in full contact with two sides of the sample chamber except for two sides thereof in contact with the electrodes, a lower surface, and an upper surface, and that has an inner space through which cooling water may circulate, and a cooling water supplier connected to the cooling water circulation channel.

### Example 3: Immunostaining Test Through Electrophoresis Technique Using Ion Conductive Film (Primary Antibody and Secondary Antibody Test)—Glial Cell Staining

Immunostaining was performed on the CLARITY sample of the brain tissue of the untransformed rat (SD Rat, 4-6 weeks old) obtained in Example 1 by using a glial fibrillary acidic protein (GFAP) antibody through an electrophoresis technique using the ion conductive film, and then was imaged.

The brain tissue sample with the diameter of 10 mm and the thickness of 1 mm prepared in Example 1 was inserted into the holder body of the sample chamber in the device prepared in Example 2, and was inserted into the biological sample fixing part to be fixed, and then each space of the electrode part and the sample chamber was filled with a borate buffer (50 mM of LiOH, 25 mM of boric acid). An antibody (1^{st} Antibody, Abcam, UK) targeting GFAP, which is a glia cell marker of the brain, in the immunostaining reagent part (antibody supply part), which is the cathode side space of both spaces of the biological sample fixing part, and an antibody (2^{nd} Antibody, Alexa-488, Abcam, UK) targeting an Fc portion of the 1^{st} antibody and labeled with the fluorescent material were infused in an amount of 1.5 μl, and were sufficiently cooled by circulating the cooling water maintained at 4° C. through the circulation channel.

Then, the power was supplied for 120 minutes while adjusting voltage and current at 50 V and 100 mA. In this case, the direction of the voltage was changed at 10 minute intervals in order to return the antibodies that passed through them to their original positions. Thereafter, the antibody was left for 30 minutes to 1 hour to be sufficiently bound to the target protein in the tissue sample. Then, a current of 100 mA was supplied in the opposite direction for 60 minutes to remove an unbound antibody.

Prior to imaging, by immersing it in FocusClear (CelExplorer Labs Co., FC-101) or an 87% Glycerol (Sigma) solution, the refractive index of the sample was adjusted to be the same as that of the lens oil of the microscope. (Refractive index=1.454.) The imaging was performed by using a confocal microscope A1 model (Nikon, Japan) and a 10× Lens (Nikon, Japan).

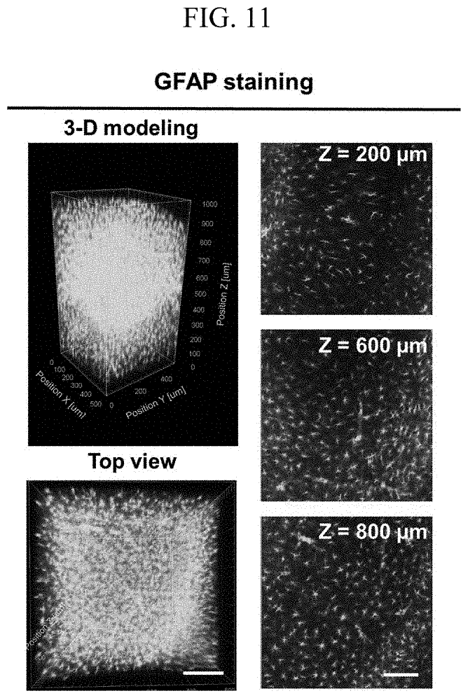

The obtained result is shown in FIG. 11 (scale bar=100 μm). As shown in FIG. 11, it can be seen that the immunostaining through the electrophoresis technique using the ion conductive film was effectively applied to the general immunostaining using the 1st antibody and the 2^{nd} antibody.

### Example 4: Immunostaining Using Lectin Reagent—Vascular Staining

Except for using a Lectin staining reagent (Lectin-594, Vector, USA) instead of the GFAP antibody, the electrophoresis method using the ion conductive film was performed in the same method as in Example 3, and the obtained result was imaged. However, the staining time took 1 hour.

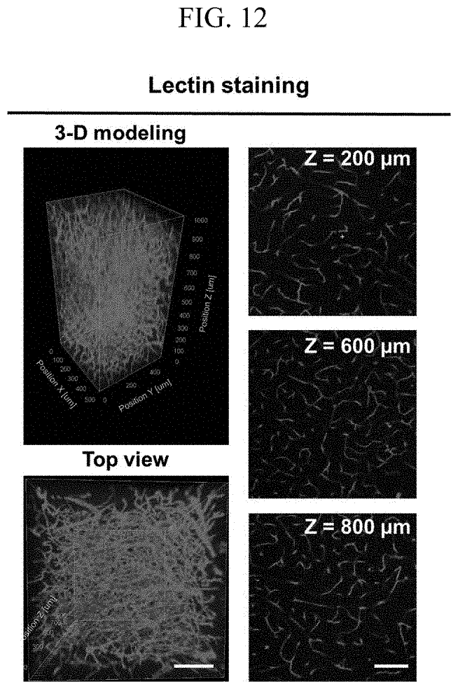

The obtained result is shown in FIG. 12 (scale bar=100 μm). As shown in FIG. 12, it can be seen that the staining was well performed to a depth of 1 mm even when the electrophoretic method using the ion conductive film was performed by using the Lectin reagent.

### Example 5: Differential Setting Test in Buffer Composition Between Sample Chamber and Outer Chamber

An electrolyte composition material lithium hydroxide (LiOH) or NaOH and a boric acid, which is appropriate for the sample at a pH of 9±0.5, was infused into the sample chamber prepared in Example 2, while an electrolyte composition material lithium hydroxide (LiOH) or NaOH and a boric acid, which is appropriate for the sample at a pH of 10.6±0.5, which is higher than that of the sample chamber, was infused into the external chamber.

In order to compare the changes in acidity when the above methods were applied, the pH changes over time were measured when using the applied voltage of 50 V and the current of 100 mA, in the same condition inside and outside the sample chamber and in the condition in which the buffer compositions were differently set, and the measured results are shown in Table

TABLE 1

| | Same composition | | Different compositions | |
|---|---|---|---|---|
| | inside and outside | | inside and outside | |
| | (comparative example) | | (embodiment) | |
| Time [h] | Inside | Outside | Inside | Outside |
| 0 | 9 | 9 | 9 | 10.6 |
| 1 | 9 | 9 | 9-10 | 10-11 |
| 2 | 9 | 9 | 9-10 | 10-11 |
| 4 | 8-9 | 9 | 9-10 | 10-11 |
| 8 | 4 | 8-9 | 9 | 10 |
| 12 | 1 | 8 | 9 | 10 |
| 16 | 1 | 7 | 9 | 10 |

Therefore, it can be seen that when the method is applied, the buffer pH in the sample chamber may be maintained at about 9 for more than 24 hours, which may be sufficient time for staining a biological sample having a diameter of 10 mm³, which is a size of the largest biological sample generally used.

### Example 6: Test of Degree of Reduction of Current According to Presence or Absence of Ion Conductive Film

In the device prepared in Example 2, the amount of current in the system was measured while changing the applied voltage, and the result is shown in Table 2 below, and as a result, the decrease in the amount of current according to the presence or absence of the ion conductive film showed a very small difference of around 2%.

TABLE 2

| | Current amount of | Current amount |
|---|---|---|
| Applied | system in state in | of system after |
| voltage | which film is attached | film is attached |
| 25 V | 47 mA | −45 mA |
| 50 V | 102 mA | −100 mA |
| 100 V | 205 mA | −200 mA |

### Example 7: Outflow Test According to Molecular Weight of Ion Conductive Film

In the device prepared in Example 2, during the electrophoresis using the staining reagent with different molecular weights, the leakage experiment of the ion conductive film was performed and the results are shown in Table 3 below, and as shown in Table 3, it was confirmed that there was no leakage from all staining reagents regardless of the molecular weights.

TABLE 3

| Type of staining reagent | 60 minutes before/after electrophoresis |
|---|---|
| Trypan blue (MW 872) | No change in concentration |
| Lectine (80 kD) | No change in concentration |
| 2nd antibody 150 kD | No change in concentration |

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

### DESCRIPTION OF SYMBOLS

| 10: | sample chamber | 12, 13: | ion conductive film |
|---|---|---|---|
| 15: | staining reagent part for biological sample | | |
| 16: | buffer part | | |
| 18: | biological sample holder | | |
| R: | staining reagent for biological sample | | |
| S: | biological sample | 21: | first electrode |
| 22: | second electrode | 24: | conductive medium |
| 24a: | buffer inlet | 24b: | buffer outlet |
| 25, 26: | film fixing plate | 28: | buffer supplier |
| 30: | magnetic body | 40: | outer chamber |

1. A biological sample staining method, comprising:
positioning a biological sample to be adjacent to a staining reagent for a biological sample, and separating the biological sample and the staining reagent for the biological sample from an outer buffer by using an ion conductive film; and
forming an electric field so that a current flows through the ion conductive film to the staining reagent for the biological sample and the biological sample,
wherein the biological sample is separated from a living body.

2. The biological sample staining method of claim 1, wherein the forming of the electric field includes forming an electric field so that a current sequentially flows in an electrode of the same polarity as the staining reagent for the biological sample, the ion conductive film, the staining reagent for the biological sample, the biological sample, and an electrode of opposite polarity to the staining reagent for the biological sample in a forward direction, a reverse direction, or both directions.

3. The biological sample staining method of claim 1, wherein the forming of the electric field includes applying a voltage to flow a current of 60 to 100 mA for 1 to 5 hours.

4. The biological sample staining method of claim 3, wherein the applying of the voltage is performed so that a direction of the current is changed at intervals of 5 to 60 minutes.

5. The biological sample staining method of claim 3, further comprising, after the applying of the voltage, leaving it for 10 minutes to 2 hours.

6. The biological sample staining method of claim 1, wherein the ion conductive film includes a cation selective permeation electrolyte membrane.

7. The biological sample staining method of claim 1, wherein the staining reagent for the biological sample is a target binding protein or a target binding nucleic acid molecule.

8. The biological sample staining method of claim 7, wherein the staining reagent for the biological sample is labeled with a fluorescent label.

9. The biological sample staining method of claim 1, wherein the biological sample is a tissue with a thickness of 0.1 mm to 10 mm.

10. The biological sample staining method of claim 1, further comprising forming a magnetic field by disposing a magnetic body at both sides of the biological sample in a direction crossing a direction in which the current flows.

11. The biological sample staining method of claim 1, wherein the biological sample staining method further includes cooling.

12. The biological sample staining method of claim 11, wherein the cooling includes
exchanging an electrode buffer,
circulating cooling water outside the ion conductive film, or
both of them.

13. A sample chamber comprising:
a sample chamber frame including a staining reagent part for a biological sample, a biological sample fixing part, and a buffer part that are arranged in a first direction in an inner space opened in the first direction;
a biological sample holder that is able to be fixed to the biological sample fixing part and is able to contain a biological sample; and
an ion conductive film that separates the inner space from the outside by being fixed to a portion of the sample chamber frame corresponding to the inner space outside the sample chamber frame.

14. The sample chamber of claim 13, wherein
the biological sample holder includes a holder body having a hole therein and a mesh positioned at both sides of the hole.

15. The sample chamber of claim 13, further comprising
a film fixing plate that presses and fixes the ion conductive film toward the sample chamber frame to a pair of sides of the sample chamber frame facing each other in the first direction.

16. The sample chamber of claim 15, wherein
the ion conductive film is fixed to the sample chamber frame by interposing a gasket for film sealing in front and rear directions according to the first direction.

17. A biological sample staining apparatus comprising:
a sample chamber of claim 13; and
an electrode part including a first electrode and a second electrode positioned outside a pair of sides of the sample chamber facing each other in the first direction.

18. The biological sample staining apparatus of claim 17, further comprising
an outer chamber into which the sample chamber is inserted into an opening that is divided into a first space and a second space by a horizontal wall and is partially opened in a middle portion of the horizontal wall,
wherein the first electrode is positioned in the first space, and the second electrode is positioned in the second space.

19. The biological sample staining apparatus of claim 17, further comprising
a buffer inlet that is connected to each of the first space and the second space of the outer chamber and is positioned at a lower end portion of the outer chamber, and a buffer outlet that is connected to the outside from each of the first space and the second space of the outer chamber and is opened upward from an upper end portion of each of the spaces.
